(19) United States
(12) Patent Application Publication
(10) Pub. No.: US 2024/0292799 A1
(43) Pub. Date: Sep. 5, 2024

(54) MEDICAL HEMP CULTIVAR CHERRY KING

(71) Applicants: KNU-INDUSTRY COOPERATION FOUNDATION, Gangwon-do (KR); CHUNCHEON BIOINDUSTRY FOUNDATION, Gangwon-do (KR)

(72) Inventors: JUNG DAE LIM, Gangwon-do (KR); YOUNG SEOK LIM, Gangwon-do (KR); BYEONG RYEOL RYU, Gangwon-do (KR); GYEONG JU GIM, Jeollabuk-do (KR); TAE HYUNG KWON, Gangwon-do (KR); JOON HEE HAN, Gangwon-do (KR)

(21) Appl. No.: 18/586,905

(22) Filed: Feb. 26, 2024

(30) Foreign Application Priority Data
Mar. 3, 2023 (KR) 10-2023-0028212

(51) Int. Cl. A01H 6/28; A01H 5/02; A61K 8/9789; A61K 36/185; A61P 31/04

(52) U.S. Cl. CPC A01H 6/28; A01H 5/02; A61K 8/9789; A61K 36/185; A61P 31/04

(57) ABSTRACT

A new hemp cultivar Cherry king (Cannabis sativa L. cv. Cherry king) plant is obtained through open pollination using Cherry wine genetic resource as a seed parent, which is deposited under Accession Number KACC 88007BP and which has a higher content of cannabinoids than the seed parent, as well as a use thereof.

### CROSS-REFERENCE TO RELATED APPLICATION AND CLAIM OF PRIORITY

This application claims the benefit under 35 USC § 119 of Korean Patent Application Nos. 10-2023-0028212 filed on Mar. 3, 2023 in the Korean Intellectual Property Office, the disclosures of which are incorporated by reference herein in their entirety.

### BACKGROUND

### 1. Technical Field

The present invention relates to new medical hemp cultivar Cherry king (Cannabis sativa L. cv. Cherry king).

This work was supported by the project of Support for Regional Research, Development, and Innovation (R&D) of Ministry of Science and ICT, South Korea (Project No: 2021-DD-UP-0379-01).

### 2. Background Art

Hemp (Cannabis sativa L.) is an annual herbaceous plant in the Cannabis genus of Cannabaceae family, and it is classified into three subspecies: C. sativa subsp. sativa, C. sativa subsp. indica, and C. sativa subsp. ruderalis. Cannabinoids, the collective term for hemp-specific terpene-derived metabolites, encompass over 113 different substances. Among the prominent cannabinoids are Δ9-tetrahydrocannabinolic acid (THCA) and cannabidiolic acid (CBDA), which are characterized by being naturally degraded into the active forms like tetrahydrocannabinol (THC) and cannabidiol (CBD) upon heat exposure. THC can induce undesirable effects such as hallucinations and possesses addictive properties. In contrast, CBD is known for its therapeutic effects like analgesic, anxiolytic, and anticonvulsant properties, making it useful as various therapeutic agents for treating medical conditions including epilepsy.

For medical purposes, hemp has been legalized in over 50 countries including Canada, several states of the United States, Australia, and Japan. Products containing CBD are also sold as functional health food products. In South Korea, a bill allowing the use of medical hemp was passed in November 2018. The Ministry of Food and Drug Safety in South Korea has approved four medicinal products containing hemp components, with Epidiolex (effective component: CBD) being primarily used as a therapeutic agent for epilepsy.

In July 2020, Andong in Gyeongsangbuk-Do, South Korea was designated as a ‘Industrial Hemp Regulation-Free Zone’, allowing for the production and processing of medical hemp. Generally, hemp is defined as having a THC content of less than 0.3% of the total dry weight of the Cannabis sativa L. plant material.

The World Health Organization (WHO) has recommended excluding medical Cannabis sativa L. from narcotic classifications, citing its very low addiction and dependence potential. The UN Commission on Narcotic Drugs also excluded Cannabis sativa L. from narcotics at the end of 2020. However, in South Korea, current laws classify all products produced from Cannabis sativa L. plant or their resin as narcotics. Accordingly, there have been concerns that if the designation of the industrial hemp regulation-free zone is not extended, the businesses may be forced to cease the operations. Meanwhile, in January 2021, there were also proposals to amend certain parts of the “Narcotics Control Act” to legalize medical Cannabis sativa L. businesses and allow for both medical and industrial uses.

Cannabis sativa L. possesses the unique characteristic of having male and female flowers on the same plant, but it can also be a dioecious plant with separate male and female flowers. Cannabis sativa L. is a heterozygous plant with cross-breeding characteristics, and when grown in open fields, genetic uniformity in the next generation cannot be guaranteed through natural cross pollination. Traditional domestic resources of Cannabis sativa L. in South Korea are primarily cultivated for fiber production, and thus mostly exhibit low levels of CBD content. Therefore, one or more embodiments of the present invention aims to develop a new Cannabis sativa L. cultivar with high levels of CBDA and/or CBD to enhance its utility for medical and industrial purposes.

Meanwhile, Korean Patent Registration No. 2346700 discloses “Method for mass-producing high levels of cannabidiol from Cannabis sativa L.”, and Korean Patent Registration No. 2269816 discloses “Composition containing hemp extract as an active ingredient for suppressing cancer, reducing anticancer side effect, and inhibiting cancer metastasis”. However, there is no mention of “new medical hemp cultivar Cherry king” as described in the present invention.

### SUMMARY

The present invention is devised under the circumstances that are described in the above. Specifically, to develop a medical hemp cultivar with high cannabidiol (CBD) content, the inventors of the present invention utilized collected overseas hemp resources and obtained F₁ plant lines through open pollination. These lines were then cultivated in greenhouses, and those with high levels of CBDA and CBD were chosen as the primary selection. Among the selected lines, CW21, a short-stem species with THC levels below 0.3% and high CBDA and CBD content, along with excellent inflorescence development property, was chosen as the secondary selection. Subsequently, the selected CW21 lines were cultivated in a smart farm, and based on cannabinoid content and morphological data obtained after harvest, the productivity per unit area (m³) was calculated. As a result, CW21-5 was identified to have the highest total CBD production amount per unit area, thus one or more embodiments of the present invention was completed. The CW21-5 plant was named Cherry king (Cannabis sativa L. cv. Cherry king).

To achieve the object described in the above, one or more embodiments of the present invention provides new hemp cultivar Cherry king (Cannabis sativa L. cv. Cherry king) plant obtained through open pollination using Cherry wine genetic resource as a seed parent, with Accession Number KACC 88007BP and a higher content of cannabinoids than the seed parent.

One or more embodiments of the present invention further provides a progeny plant derived from the above new hemp cultivar Cherry king plant.

One or more embodiments of the present invention further provides a feminized seed obtained by induction of male flowers on female plant of the above new hemp cultivar Cherry king plant, as well as hempseed oil and seed protein derived from the feminized seed.

One or more embodiments of the present invention further provides a F₁ hemp plant produced by crossing the new hemp cultivar Cherry king plant with other hemp cultivar, and a seed of the F₁ hemp plant.

One or more embodiments of the present invention further provides hempseed oil and a seed protein derived from the seed of the F₁ hemp plant.

One or more embodiments of the present invention further provides a transgenic hemp plant obtained by transformation of the new hemp cultivar Cherry king plant.

One or more embodiments of the present invention further provides a mutant plant obtained by treating the new hemp cultivar Cherry king plant with mutagen.

One or more embodiments of the present invention still further provides a functional health food composition, a pharmaceutical composition, an antimicrobial composition, and a cosmetic composition comprising an extract of the new hemp cultivar Cherry king plant as effective component.

The new hemp cultivar Cherry king according to one or more embodiments of the present invention is an excellent medical hemp with high levels of cannabinoid components showing medical benefits, and, as it exhibits high cannabinoid productivity even when cultivated in smart farms, it is expected to be advantageously utilized in related industrial sectors.

### BRIEF DESCRIPTION OF THE DRAWINGS

FIG. 1 shows the inflorescence and leaf portion at the branch end of Cherry king (line name: CW21-5).

FIG. 2 shows the young plant of Cherry king (line name: CW21-5) that can be successfully cultivated and propagated under in vitro conditions.

FIGS. 3A to 3D illustrate the results of cannabinoid content analysis of plants of CW21 lines cultivated in smart farm, in which the analysis was made by date. The specifics are as follows; (FIG. 3A): total CBD in leaves, (FIG. 3B): total CBD in inflorescence, (FIG. 3C): total Δ9-THC in leaves, and (FIG. 3D): total Δ9-THC in inflorescence.

FIG. 4 is a heatmap of the Pearson correlation coefficient matrix between morphology and chemotype.

FIG. 5 shows the results of principal component analysis (PCA) and self-organizing clustering (SOM) between morphology and chemotype.

FIGS. 6A to 6D are graphs for analyzing the apoptotic effect of an extract of Cherry king (line name: CW21-5) or Chungsam cultivar on lung cancer cell line (A549).

FIG. 7 is a graph for analyzing the activity of inhibiting microbial growth by an extract of Cherry king (line name: CW21-5) or Chungsam cultivar.

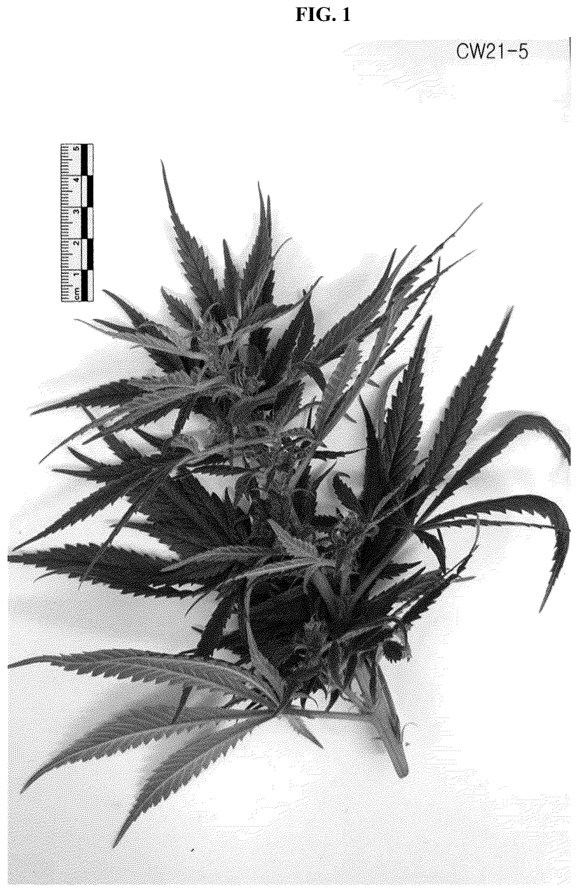

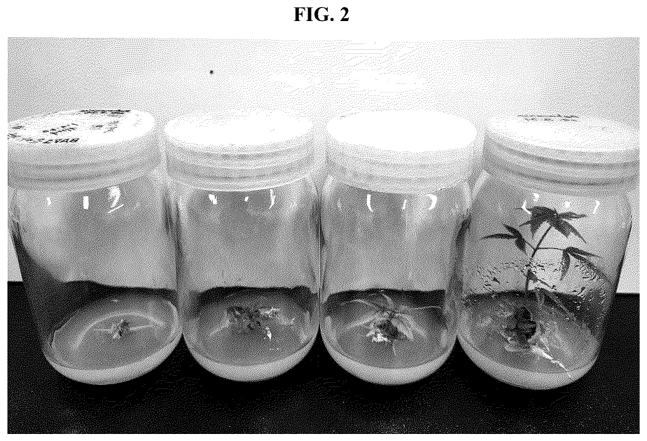

### DETAILED DESCRIPTION

To achieve the purpose of the present invention, one of more embodiments of the present invention provides new hemp cultivar Cherry king (Cannabis sativa L. cv. Cherry king) plant obtained through open pollination using Cherry wine genetic resource as a seed parent(female), with Accession Number KACC 88007BP and a higher content of cannabinoids than the seed parent.

The Cannabis sativa L. cv. Cherry king was duly deposited with the Korean Agricultural Culture Collection (KACC) (having the address of 166 Nongsaengmyeon-ro, 1 seo-myeon, Wanju-gun, Jeollabuk-do 55365, 55365, Republic of Korea) under the Access number of KACC 88007BP on Feb. 10, 2023. The deposit has been made under the terms of the Budapest Treaty and all restrictions imposed by the depositor on the availability to the public of the biological material will be irrevocably removed upon the granting of a patent.

One of more embodiments of the present invention further provides a progeny plant derived from the above new hemp cultivar Cherry king plant.

With regard to the Cherry king plant according to one of more embodiments of the present invention, the cannabinoids whose content is higher than that of the seed parent may be cannabidiolic acid (CBDA) and cannabidiol (CBD), but they are not limited thereto. The CBDA is decomposed into CBD by heat.

The genetic resource of Cherry wine hemp used as the seed parent in the present invention is an overseas hybrid having genetic composition of C. sativa subsp. indica:C. sativa subsp. sativa=50:50 (%), and it is a hemp with higher THC (tetrahydrocannabinol) content than CBD content.

The Cherry king ‘plant’ of the present invention includes both the whole plant or part of the plant. The ‘part’ of the plant according to the present invention may include, although not limited thereto, inflorescences, leaves, roots, stems, or the like of the new hemp cultivar Cherry king. Additionally, the progeny plant derived from the Cherry king plant may be a seedling-type progeny plant derived through asexual reproduction of the new hemp cultivar Cherry king, but it is not limited thereto. The asexual reproduction method may include, but is not limited to, plant tissue culture techniques such as cell culture, protoplast culture, chemical culture, and callus culture.

In the case of hemp, due to the nature of the species, it is divided into female plant and male plant, or both male and female flowers appear on the same plant at the same time. Thus, in order to maintain the variety characteristics, male and female flowers are grown separately and the female flowers are chemically treated to produce female seeds, or the hybrid F₁ generation line is cultivated so that the excellent lines can be maintained through vegetative propagation.

Through the cultivation based on nodal culture or cutting and planting, the new hemp cultivar Cherry king according to the present invention can be maintained as the plant itself without any genetic mutation.

By using Cherry wine genetic resource as seed parent, the inventors of the present invention obtained, through open pollination, a hemp line with higher cannabinoid content in leaves and inflorescences compared to the seed parent, and named the line “Cherry king (Cannabis sativa L. cv. Cherry king)”. Then, its feminized seeds were taken as representative sample and deposited in the Korean Agricultural Culture Collection (KACC) on Feb. 10, 2023 (Accession Number: KACC 98135P), and an international deposit number was assigned on Dec. 13, 2023. (Accession Number: KACC 88007BP).

One of more embodiments of the present invention further provides a feminized seed obtained by induction of male flowers on female plant of the above new hemp cultivar Cherry king plant, as well as hempseed oil and seed protein derived from the feminized seed.

The sex of hemp is determined by X and Y sex chromosomes of a pollen. Pollen of a male plant consists of female pollen with n=9+X and male pollen with 9+Y. The stigma plant pollinated with female pollen becomes a female plant and the stigma plant pollinated with male pollen becomes a male plant. As such, if the sex of the female plant, which normally has somatic sex chromosome composition of XX, is altered to induce male traits, the pollen from this plant will contain only X chromosomes. By cross-pollinating this with a regular female plant, the resulting seeds, modified in this manner, will become ‘feminized seeds’ that yield only females with 100% certainty. This can significantly enhance the efficiency of breeding and seed collecting.

In one of more embodiments of the present invention, the induction of male traits in the aforementioned female hemp plant can be accomplished through methods that are well known in the pertinent art, such as change in daylight hours or treatment with silver nitrate during the vegetative growth stage.

Hemp seeds (i.e., achenes) contain a large amount of unsaturated fatty acids, and hemp oil, in particular, contains γ-linolenic acid (GLA), and thus showing preventive or therapeutic effects on skin aging, atopic dermatitis, cardiovascular diseases, rheumatoid arthritis, and others.

One of more embodiments of the present invention further provides a F₁ hemp plant produced by crossing the new hemp cultivar Cherry king plant with other hemp cultivar, as well as a seed of the F₁ hemp plant. The crossing may be performed according to methods related to plant breeding that are known to those skilled in the pertinent art and, since the new hemp cultivar Cherry king according to the present invention is characterized by having a high content of cannabinoid components with medical efficacy, the Cherry king plant can be used as a seed parent material for breeding new varieties of hemp, especially medical hemp, but it is not limited to this. The F₁ hemp plant includes the whole plant, part of the plant, and progeny plant derived therefrom.

One of more embodiments of the present invention further provides hempseed oil and a seed protein derived from the seed of the F₁ hemp plant.

One of more embodiments of the present invention further provides a transgenic hemp plant obtained by transformation of the new hemp cultivar Cherry king plant, as well as a transgenic seed of the transgenic hemp plant. The transformation method can be accomplished through various techniques that are known in the pertinent art, and the transgenic hemp plant according to one of more embodiments of the present invention includes all transgenic plants having the genetic background of Cherry king plant.

One of more embodiments of the present invention further provides a mutant plant obtained by treating the new hemp cultivar Cherry king plant with mutagen. The mutagen includes physical mutagens such as X ray, γ ray (⁶⁰Co), β ray (³²P, ³⁵S, or the like) and neutron beam and chemical mutagens such as proflavin, acridine orange, N-methyl-N′-nitro-N-nitrosoguanidine (NTG or MNNG), 4-nitroqyinoline 1-oxide (4-NQO), nitrous acid (HNO₂), hydroxylamine (NH₂OH), dimethylsulfate (DMS), diethylsulfate (DES), ethyl ethanesulfonate (EES), methyl methanesulfonate (MMS), and ethyl methanesulfonate (EMS), but is not limited thereto.

One of more embodiments of the present invention further provides a functional health food composition including an extract of the new hemp cultivar Cherry king plant as effective component.

With regard to the functional health food composition according to one of more embodiments of the present invention, the new hemp cultivar Cherry king is, as a medical hemp, characterized by having a high content of medically beneficial cannabinoid compounds (CBD, CBDA). The extract of the Cherry king may be obtained by extracting a part, a seed, or the like of the Cherry king plant with an extraction solvent selected from the group consisting of water, C₁ to C₆ lower alcohols, and mixtures thereof, but it is not limited thereto. Additionally, the extraction method may utilize all conventional methods that are well known in the art, including filtration, hot water extraction, immersion extraction, reflux cooling extraction, and ultrasound extraction.

The term “extract” used in this specification generally has a meaning that is commonly known in the pertinent art as a crude extract, but, in a broad sense, it also encompasses fractions that are obtained by further fractionating the extract. In other words, the extract includes not only those obtained using the aforementioned extraction solvents but also those obtained by applying additional purification processes. For instance, fractions obtained by additionally performing various purification processes such as passing the aforementioned extract through an ultrafiltration membrane having certain molecular weight cut-off or separations performed by various chromatography techniques (designed for separation based on size, charge, hydrophobicity, or affinity) are also included in the extract of the present invention.

The functional health food composition of one of more embodiments of the present invention is preferably manufactured in any one of the formulation forms selected from powder, granules, pill, tablet, capsule, candy, syrup, and beverage, but it is not limited thereto.

When using the functional health food composition of one of more embodiments of the present invention as a food additive, the extract of the new hemp cultivar Cherry king can be added directly or used in combination with other foods or food ingredients, and it can be appropriately used according to common methods. The amount of the active ingredient can be appropriately determined depending on their intended use (e.g., prevention, health, or therapeutic treatment). Generally, when manufacturing food or beverages, the composition of one of more embodiments of the present invention is added to the raw materials in an amount of not more than 15 parts by weight, preferably not more than 10 parts by weight. However, in cases of long-term consumption for health and hygiene purposes or for managing health, the amount may be less than the aforementioned range, and, since there may be no problem with safety, the active ingredient may also be used in an amount exceeding the aforementioned range.

One of more embodiments of the present invention further provides a pharmaceutical composition including an extract of the new hemp cultivar Cherry king plant as effective component.

With regard to the pharmaceutical composition according to one of more embodiments of the present invention, the new hemp cultivar Cherry king is, as a hemp for medical use, characterized by having a high content of cannabinoid components known for their medical efficacy. The extract of the Cherry king is as described above.

According to Project CBD, at least 50 symptoms are considered to be improved by cannabidiol (CBD). These symptoms include pain, seizures, muscle spasms, nausea caused by chemotherapy, digestive disorders such as colitis and inflammatory bowel disease, muscle tension, epilepsy, multiple sclerosis, Parkinson's disease, mood disorders, anxiety, post-traumatic stress disorder, and high blood pressure.

The pharmaceutical composition of one of more embodiments of the present invention may further include, other than the effective component described above, a pharmaceutically acceptable carrier, vehicle, or diluent, and it can take various forms for oral or parenteral administration. When formulated, production is made by using commonly-used diluents or vehicles such as fillers, expanders, binders, wetting agents, disintegrants, or surfactants. For solid preparations intended for oral administration, they may include capsules, powders, granules, tablets, pills, etc., and these solid formulations are formulated by mixing one or more compounds with at least one vehicle such as starch, calcium carbonate, sucrose, lactose, gelatin, etc. Additionally, lubricants such as magnesium stearate, talc, etc. can be used in addition to simple vehicles. For liquid preparations intended for oral administration, they may include suspensions, emulsions, syrups, aerosols, etc., and in addition to commonly used simple diluents like water and liquid paraffin, various vehicles such as wetting agents, sweeteners, flavors, preservatives, etc. may be included. For preparations intended for parenteral administration, they may include sterilized aqueous solutions, non-aqueous solvents, suspensions, emulsions, freeze-dried formulations, suppositories, etc. Examples of non-aqueous solvents and solvents for suspension include propylene glycol, polyethylene glycol, vegetable oils such as olive oil, injectable esters such as ethyl oleate, etc. Examples of suppository bases include Witepsol, Macrogol, Tween 61, cocoa butter, laurin, glycerol gelatin, etc. When the pharmaceutical composition is administered parenterally, it is preferable to choose a method like topical application on skin, or an intraperitoneal, rectal, intravenous, intramuscular, subcutaneous, intrathecal, and intracerebrovascular injection method.

The pharmaceutical composition according to one of more embodiments of the present invention is administered in a pharmacologically effective amount. In one of more embodiments of the present invention, the expression “pharmacologically effective amount” refers to an amount sufficient to treat a disease with a reasonable benefit/risk ratio applicable to medical therapy. The effective dosage level can be determined based on various factors, including the type and severity of the patient's condition, the activity of the drug, sensitivity to the drug, administration time, route of administration, elimination rate, treatment duration, factors including other drugs that are simultaneously used, and other factors well-known in the medical field. The pharmaceutical composition of one of more embodiments of the present invention can be administered as an individual therapeutic agent or in combination with other therapeutic agents, sequentially or simultaneously with conventional therapies, and in a single dose or multiple doses. It is important to administer, with consideration of the aforementioned factors, the minimum effective amount that can achieve maximum efficacy without having any adverse effects, and this can be readily determined by a person who is skilled in the pertinent art.

One of more embodiments of the present invention further provides an antimicrobial composition including an extract of the new hemp cultivar Cherry king plant as effective component. The aforementioned antimicrobial property may extend to antimicrobial property against Escherichia coli and Streptococcus mutans, but is not limited thereto.

One of more embodiments of the present invention still further provides a cosmetic composition including an extract of the new hemp cultivar Cherry king plant as effective component. The aforementioned cosmetic composition can be preferably manufactured in any of the formulations selected from solution, suspension, emulsion, paste, gel, cream, lotion, powder, soap, surfactant-containing cleansing agent, oil, powder foundation, emulsion, foundation, wax foundation, and spray, among others. However, it is not limited thereto.

In the case of the cosmetic composition of one of more embodiments of the present invention being in the formulation form of a solution or emulsion, solvents, solvent vehicles, or emulsifying agents are used as a carrier component. For example, water, ethanol, isopropanol, ethyl carbonate, ethyl acetate, benzyl alcohol, benzyl benzoate, propylene glycol, 1,3-butylene glycol oil, glyceryl fatty esters, polyethylene glycol, or sorbitan fatty acid esters can be used.

In the case of the cosmetic composition of one of more embodiments of the present invention being in the formulation form of a suspension, water, ethanol, or propylene glycol as liquid diluents, ethoxylated isostearyl alcohol, polyoxyethylene sorbitol esters, and polyoxyethylene sorbitan esters as suspending agents, and microcrystalline cellulose, aluminum hydroxide, bentonite, agar, or tragacanth can be used.

In the case of the cosmetic composition of one of more embodiments of the present invention being in the formulation form of a paste, cream, or gel, animal fibers, plant fibers, waxes, paraffin, starch, tragacanth, cellulose derivatives, polyethylene glycol, silicone, bentonite, silica, talc, or zinc oxide may be used as a carrier component. In the case of the cosmetic composition of one of more embodiments of the present invention being in the formulation form of powder or spray, lactose, talc, silica, aluminum hydroxide, calcium silicate, or polyamide powder can be used as a carrier component. Especially in the case of a spray, a propellant such as chlorofluorohydrocarbons, propane-butane, or dimethyl ether may be additionally included.

In the case of the cosmetic composition of one of more embodiments of the present invention being in the formulation form of a surfactant-containing cleansing agent, fatty alcohol sulfates, fatty alcohol ether sulfates, sulfosuccinic acid monoester, acethionate, imidazolinium derivatives, methyltaurates, sarcosinates, fatty acid amide ether sulfates, alkylamidobetaines, fatty alcohols, glyceride fatty acids, diethanolamide fatty acids, vegetable oils, lanolin derivatives, or ethoxylated glyceryl fatty acid esters can be used as a carrier component.

Hereinbelow, one of more embodiments of the present invention are explained in greater detail in view of Examples. However, the following Examples are given only for exemplification of the present invention and it is evident that the scope of the present invention is not limited by them.

### EXAMPLES

### Experimental Example 1. Determination of Cannabinoid Content

An embodiment of the present invention was carried out under the approval of the Narcotics Handler (i.e., academic researcher) (Approval Number: Seoul-1770) and Narcotics and Raw Material Handling Approval (Narcotics Policy Department-4789, Narcotics Policy Department-6373, Ministry of Food and Drug Safety, South Korea).

The samples for cannabinoid content analysis were freeze-dried samples, which were stored at 4° C. until extraction. As CBD and A9-THC standards, products from CAYMAN (CAYMAN Co., USA) were used. As a CBDA standard, a product from Cerilliant (Round Rock, USA) was used, and as a A9-THCA standard, a product from Sigma-Aldrich was used. Each standard reagent was diluted to concentrations of 10, 20, 30, 40, and 50 ppm to construct a standard curve. The measured values of the samples were then fitted to the standard curve to calculate the content of each cannabinoid.

After completing the freeze-drying process, 1 g of the sample was weighed and placed in a 50 ml conical tube. Then, 30 ml of methanol with a purity of at least 99.5% were added, followed by sonication using an ultrasonic homogenizer (JEIO TECH Co., Ltd., Korea) at room temperature (25±1° C.) for 30 minutes. The supernatant was then filtered using a 0.45 μm syringe filter and used for HPLC analysis. The content unit (%) of each cannabinoid was expressed as ratio of each cannabinoid relative to dry weight of the corresponding sample.

TABLE 1
HPLC operating condition
for the analysis of CBDA, THCA, CBD and THC

| | |
|---|---|
| Instrument | Shimadzu LC-20AT HPLC system |
| Column | Reverse phase Zorbax SB-C₁₈ |
| | (4.6 mm * 100 mm, 3.5 μm) |
| Detector | UV-VIS detector (275 nm) |
| Solvent | 70% Acetonitrile containing |
| | 0.1% phosphoric acid |
| Flow rate | 1.5 ml/min |
| oven | 27° C. |
| Injection volume | 10 μl |
| Elution type | Isocratic |

### Experimental Example 2. In Vitro Introduction of Hemp Material

### 2-1. Preparation of Culture Medium

MS medium (Duchefa Biochemie, Haarlem, Netherlands) was taken in an amount of 4,405.19 mg and dissolved in 900 ml of distilled water. Thirty grams of sucrose (suitable for plant cell culture, Sigma-Aldrich Co., USA) were added thereto. Then, Meta-topolin (Duchefa Biochemie, Netherlands) was added such that it has a final concentration of 0.2 μM. After stirring, pH was adjusted to 5.75 using 1 N sodium hydroxide (DAEJUNG chemicals and metals Co., Ltd., Korea). After adjusting the volume to 1,000 ml, 4% of plant agar (Duchefa Biochemie) was additionally added. After that, the mixture was heated on a hot plate with stirring until it boils. The resultant was aliquoted in an amount of 50 ml to culture vessels and then subjected to autoclave under moisture condition at 151,988 Pa and 121° C. for 15 minutes. The resultant was allowed to solidify at room temperature (22±2° C.).

### 2-2. In Vitro Introduction of Hemp Explant

The inside of a clean bench was sterilized with an ultraviolet lamp and then sterilized again with 70% ethanol (DAEJUNG chemicals and metals Co., Ltd.). Nodes including the young leaves at the bottom part of the hemp were collected. They were transferred to a clean bench and placed in a sterilized glass bottle. Then, 70% ethanol was added and stirred for 30 seconds. After removing the ethanol and adding 30 ml of 2% sodium hypochlorite (Junsei Chemical Co., Ltd., Japan), polysorbate 20 (tween 20, Junsei Chemical Co., Ltd.) was added to the final concentration of 0.08% to 0.12%. Subsequently, washing was carried out by stirring for 15 minutes. After removing the solution, the resultant was washed six times with sterile distilled water. On a sterile Petri dish, the top part of the leaf was cut to have one node. After placing it on a culture medium, it was cultivated under LED light conditions of 18 h/day and temperature conditions of 23±2° C. One month later, the regenerated in vitro plants were allowed to propagate in the same culture medium.

### Example 1. Crossing Process

The inventors of the present invention collected overseas genetic resources of hemp which has been cultivated for medical and hemp cloth purposes, and evaluated its botanical and cultural characteristics. All collected samples of overseas hemp genetic resources are stored in the Department of Herbal Medicine Resources Development at Kangwon National University.

TABLE 2
Collection of overseas native genetic resources and
determination of cannabinoid content

| Country of origin | THC (%) | CBD (%) | Note |
|---|---|---|---|
| India | 0.53 | 1.19 | |
| UK 1 | 0.01 | 0.11 | |
| UK 2 | 0.13 | 1.44 | |
| Spain | — | — | Not germinated |
| Latvia | 0.01 | 0.19 | |
| Singapore | — | — | Not germinated |
| Portugal | 0.01 | 0.19 | |
| France | — | — | Not germinated |
| Lithuania | 0.01 | 1.16 | |

TABLE 3
Collection of overseas hybrid species
and determination of cannabinoid content

| | | THC | CBD |
|---|---|---|---|
| Name of the species | Genetic composition (%) | (%) | (%) |
| PURPLE AFGHAN KUSH | Indica dominant | 15-21 | 0 |
| SHARK SHOCKED CBD | Indica:sativa = 80:20 | 6 | 6 |
| (100% DNF) | | | |
| DINAMED CBD | Indica:sativa = 40:60 | 0.4-0.6 | 10-14 |
| (100% DNF) | | | |
| QUICK GORILLA | Sativa | — | — |
| GORILLA AUTO | Indica:ruderalis = 50:50 | 19 | 0.2 |
| (100% DNF) | | | |
| MOBY DICK XXL AUTO | sativa (100%) | 15 | 0 |
| (100% DNF) | | | |
| QUICK DINAMED CBD | Indica:sativa = 45:55 | 0.5-0.8 | 14-18 |
| (100% DNF) | | | |
| MOBY DICK (100% DNF) | Indica:sativa = 40:60 | 17-22 | 0 |
| CHEESE CBD (AUTO), | Indica:sativa = 50:50 | 8-10 | 10-12 |
| 100% DNF | | | |
| DINAMED CBD PLUS | Indica:sativa 40:60 | 0.5-1 | 15-20 |
| (100% NF) | | | |
| BLUE DREAM CBD | Indica:sativa = 3:65 | 10 | 10 |
| Alpine Star CBD Feminized | Indica:sativa = 50:50 | 0.3 | 20 |
| (KHV1) | | | |
| CBD Paqle Kush Auto | Indica:Sativa:Ruderalis = | 0.5 | 17 |
| flowering (KHV2) | 70:10:20 | | |
| Charlotte's Web ♀ Seeds | Sativa dominant | 0.3 | 10 |
| (KHV3) | | | |
| Harlequin ♀ seeds (KHV4) | Sativa dominant | 6.5 | 9.1 |
| MAZAR Auto flowering | Indica:Sativa:Ruderalis = | 10 | 0.2 |
| feminized (KHV5) | 50:30:20 | | |
| Cherry wine | Indica:Sativa = 50:50 | 15 | 0.3 |

TABLE 4
Collection of genetic resources from Korean Rural Development
Administration and determination of germination rate

| Serial Number | IT Number | Origin | Germination rate |
|---|---|---|---|
| RDA-1 | 102837 | KOR | 0 |
| RDA-2 | 104521 | KOR | 0 |
| RDA-3 | 105982 | KOR | 0 |
| RDA-4 | 109149 | KOR | 0 |
| RDA-5 | 162890 | KOR | 40 |
| RDA-6 | 176019 | KOR | 56 |
| RDA-7 | 176020 | KOR | 22 |
| RDA-8 | 177143 | KOR | 56 |
| RDA-9 | 160386 | KOR | 0 |
| RDA-10 | 180387 | KOR | 0 |
| RDA-11 | 180388 | KOR | 0 |
| RDA-12 | 180389 | KOR | 0 |
| RDA-13 | 180390 | KOR | 44 |
| RDA-14 | 180391 | KOR | 53 |
| RDA-15 | 183201 | KOR | 86 |
| RDA-16 | 102837 | KOR | 52 |
| RDA-17 | 104521 | POR | 35 |
| RDA-18 | 105983 | CHN | 8 |
| RDA-19 | 199568 | CHN | 34 |
| RDA-20 | 199569 | CHN | 40 |
| RDA-21 | 199570 | CHN | 37 |
| RDA-22 | 199571 | CHN | 34 |
| RDA-23 | 199572 | KAZ | 0 |
| RDA-24 | 199573 | AFG | 10 |
| RDA-25 | 200318 | NPL | 0 |
| RDA-26 | 207757 | YUG | 5 |
| RDA-27 | 207758 | YUG | 30 |
| RDA-28 | 207759 | YUG | 30 |
| RDA-29 | 207760 | YUG | 18 |
| RDA-30 | 207761 | EST | 30 |
| RDA-31 | 207762 | UNK | 20 |
| RDA-32 | 207763 | RUS | 0 |
| RDA-33 | 207764 | RUS | 34 |
| RDA-34 | 207765 | RUS | 30 |
| RDA-35 | 207766 | RUS | 4 |
| RDA-36 | 207767 | RUS | 15 |
| RDA-37 | 207768 | INK | 10 |
| RDA-38 | 207769 | INK | 23 |
| RDA-39 | 207810 | INK | 0 |
| RDA-40 | 209078 | RUS | 0 |
| RDA-41 | 209079 | RUS | 0 |
| RDA-42 | 209080 | UNK | 21 |
| RDA-43 | 209081 | RUS | 0 |
| RDA-44 | 209082 | RUS | 0 |
| RDA-45 | K109453 | RUS | 66 |
| RDA-46 | K109502 | RUS + KJI | 8 |
| RDA-47 | K109510 | RUS + KJI | 0 |
| RDA-48 | K133756 | BGR | 0 |
| RDA-49 | K151644 | KOR | 4 |
| RDA-50 | K153762 | PRK | 0 |
| RDA-51 | K153763 | PRK | 0 |

The male hemp seeds in Tables 2 to 4 including the pollen parent-line Cherry wine cultivar were sown in seed trays (54 cm×28 cm, 72 well) containing a mixture of bio-soil (Heungnong Jongmyo Co., Korea) and top-soil (Taeheung F&G, Korea) in a 2:1 ratio. After 2 weeks, they were transplanted into larger pots (diameter 50 cm×depth 70 cm) and cultivated for 8 weeks in a greenhouse with adjustable light conditions (18 h/day light condition, 25±3° C.). After 8 weeks, the light condition was adjusted to 12 h/day, and natural pollination was facilitated with the Cherry wine seed parent cultivar. It was possible to harvest mature F₁ seeds after approximately 6 weeks following the pollination. Mature hemp achenes exhibit a dark brown color, and depending on the variety, may have a marbled pattern. Immature achenes appear greenish and are prone to breakage, with very weak or no germination ability when sown.

The F₁ generation hemp seeds produced through open pollination numbered approximately 1,000. After sowing in seed trays with a mixture of bio-soil and top-soil in a 2:1 ratio as described in the above, around 100 successfully germinated plants were transplanted into an artificial nutritional cultivation environment under LED conditions after 2 weeks. Among them, 60 plants survived successfully under the artificial light and nutritional media cultivation conditions. After 60 days, the light conditions were changed to 12 h/day, and all the male plants and inferior lines that did not adapt to the environment and failed to grow were removed. Subsequently, 16 female plants exhibiting the characteristics of short stem height like not more than 1 m, which is suitable for artificial cultivation environments, and bushy growth forms were selected (specifically, they were designated as CW21-1 to CW21-16), and then the secondary selection was carried out as follows.

### Example 2. Secondary Selection of Selected Smart Farm-Exclusive Line

During the cultivation in smart farm, the nutrient media solution composition was as described in Table 5, and LED conditions were maintained with the following parameters: brightness at 40,076±307 lux, PPFD at 692.4, PFD-R at 291.2, PFD-G at 254.8, and PGF-B at 146.5. For the initial 60 days, the plants were induced for vegetative growth under a 16-hour/day lighting scheme. On the 60th day, the lighting duration was changed to 12 hours/day (short-light treatment) to induce flowering.

TABLE 5
Composition of nutrient media solution used
for smart farm cultivation of 18-1 lines

| | A solution | | B solution | | |
|---|---|---|---|---|---|
| Nutrient name | (50 L) | | (50 L) | | |
| Ca(NO₃)•4H₂O | 1.50 | kg | | | A solution |
| KNO₃ | 3.79 | kg | 3.79 | kg | and B |
| | | | | | solution were |
| (NH₄)₂HPO₄ | | | 1.60 | kg | subjected to |
| MgSO₄ | | | 4.30 | kg | mixing to maintain |
| Fe-EDTA | 460.0 | g | | | an EC range |

| | | | |
|---|---|---|---|
| MnSO₄ | 30.8 | g | between 1.2 to 1.7 |
| H₃BO₃ | 57.2 | g | (dSm⁻²), pH 6.0. |
| ZnSO₄ | 3.6 | g | |
| CuSO₄ | 1.3 | g | |
| (NH₄)₆Mo₇O₂₄•4H₂O | 0.4 | g | |

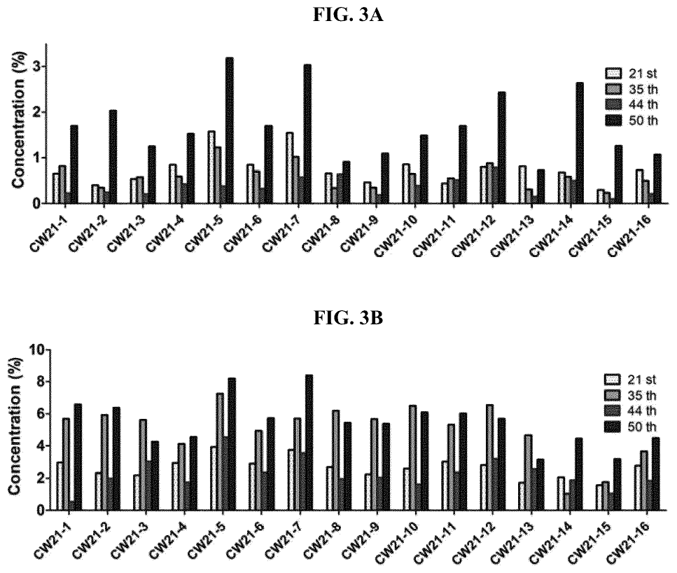

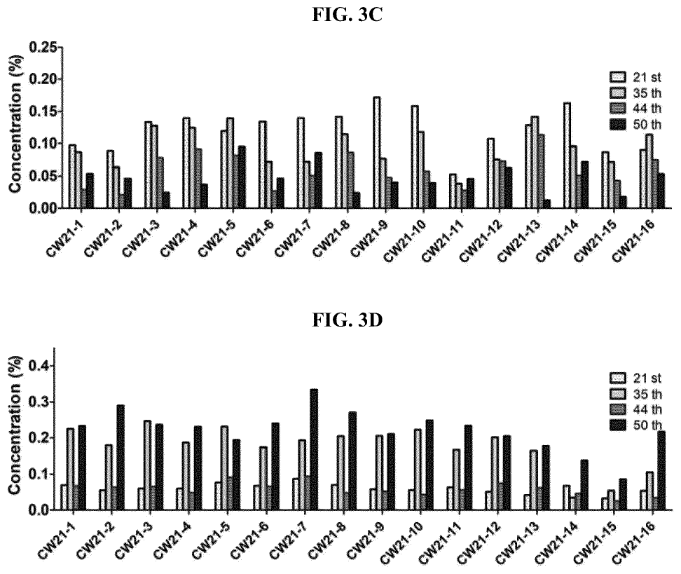

After flowering, the analysis of total CBD was carried out. As a result, a similar cannabinoid trend was revealed across the all lines. The total CBD content in the leaves was 0.7586±0.3549% on day 21 after flowering treatment, 0.6061±0.2747% on day 35 after flowering treatment, 0.3659±0.1962% on day 44 after flowering treatment and peaked at 1.7316±0.7442% on day 50 after flowering (FIG. 3A). The total CBD content in the inflorescences (i.e., cluster of flowers) showed an increase from 2.6513±0.6371% on day 21, reaching 5.0402±1.6898% on day 35, then decreasing to 2.2593±0.9702% on day 44, and finally peaking at 45.166±45.66% on day 50 (FIG. 3B). The total Δ9-THC content in the leaves exhibited a slightly different pattern compared to the inflorescences with values of 0.1221±0.0325% on day 21, 0.0956±0.0306% on day 35, 0.0594±0.0270% on day 44, and 0.0471±0.0232% on day 50 (FIG. 3C). The total Δ9-THC content in the inflorescences was 0.0607±0.0213% on day 21, 0.1747±0.0607% on day 35, 0.0583±0.0185% on day 44, and 0.2214±0.0579% on day 50 (FIG. 3D). Taken together, the day with the lowest total Δ9-THC content and the highest CBD content in the leaves was day 50 and the CBD content in the inflorescences was highest on day 50, and thus the harvest time was set to day 50 after the short-daylight treatment. Morphological data were also collected at the selected harvest time (Table 6).

TABLE 6
Morphological data collected during harvest period of CW21
line cultivated with smart farm nutrient media solution

| | Plant | Leaf | Leaf | | Stem | | | petiole | Stem | Whole plant |
|---|---|---|---|---|---|---|---|---|---|---|
| Plant | height | length | width | Branch | diameter | Inf. | Inf. | color | color | diameter |
| line | (cm) | (cm) | (cm) | number | (mm) | number | size¹⁾ | (Red %) | (Red %) | (cm) |
| CW21-1 | 44.1 | 18 | 3.2 | 12 | 15.40 | 198 | ++ | 60 | 5 | 66 |
| CW21-2 | 52.5 | 12 | 3.0 | 10 | 8.62 | 320 | +++ | 95 | 75 | 68 |
| CW21-3 | 50.2 | 23 | 5.1 | 26 | 14.86 | 76 | + | 15 | 5 | 37 |
| CW21-4 | 84.0 | 14 | 2.5 | 23 | 10.44 | 146 | ++ | 50 | 5 | 50 |
| CW21-5 | 68.2 | 15 | 3.1 | 19 | 13.06 | 205 | ++++ | 30 | 5 | 43 |
| CW21-6 | 69.3 | 12 | 1.4 | 20 | 7.04 | 330 | ++ | 40 | 50 | 58 |
| CW21-7 | 78.9 | 14 | 1.8 | 20 | 10.50 | 460 | +++ | 40 | 20 | 78 |
| CW21-8 | 38.5 | 12 | 1.5 | 12 | 10.09 | 248 | ++ | 5 | 0 | 87 |
| CW21-9 | 62.1 | 11 | 1.3 | 17 | 13.27 | 512 | ++++ | 5 | 0 | 90 |
| CW21-10 | 47.0 | 19 | 2.1 | 14 | 8.15 | 245 | ++++ | 85 | 80 | 70 |
| CW21-11 | 72.0 | 13 | 2.0 | 14 | 6.84 | 179 | ++ | 80 | 80 | 70 |
| CW21-12 | 54.2 | 15 | 1.8 | 16 | 8.51 | 295 | ++++ | 75 | 60 | 73 |
| CW21-13 | 59.0 | 15 | 2.8 | 14 | 11.06 | 349 | ++ | 15 | 0 | 50 |
| CW21-14 | 61.2 | 10 | 1.2 | 11 | 8.39 | 300 | ++++ | 30 | 30 | 89 |
| CW21-15 | 88.9 | 19 | 4.0 | 17 | 11.23 | 201 | ++ | 30 | 0 | 58 |
| CW21-16 | 69.3 | 18 | 3.1 | 15 | 7.51 | 85 | ++ | 40 | 0 | 68 |

¹⁾+; very small, ++; small, +++; big, ++++; very big.

Because the smart farm facility based on cultivation using nutrient media solution is more spatially efficient than outdoor cultivation, calculating the production amount is essential for medical hemp for producing CBD. Based on the collected cannabinoid content and morphological data described in the above, the production amount per cubic meter (m³) was calculated according to the following formula.

$$CBD + CBDA production per cubic meter ( μg ) = \frac{a \times b \times 1 , 000 , 000}{plant {width}^{2} \times plant height}$$

a, CBD+CBDA concentration (ppm; μg/g)
b, dry weight of leaf or inflorescence (g)
* width and height are in centimeters

As a result, CW21-5 was found to exhibit the highest CBD production amount per unit meter at 53.078±0.242 grams (Table 7).

TABLE 7
CBD Production amount per cubic meter by CW21 lines

| | Total CBD content (g, CBD + CBDA) | | | | | |
|---|---|---|---|---|---|---|
| Plant name | Leaf | | Inflorescence | | Total | |
| CW21-1 | 7.961 ± | 0.142^{d} | 30.536 ± | 0.107^{b} | 38.498 ± | 0.248^{b} |
| CW21-2 | 1.838 ± | 0.017^{g} | 11.871 ± | 0.003ⁱ | 13.709 ± | 0.014^{l} |
| CW21-3 | 8.353 ± | 0.114^{c} | 21.341 ± | 0.176^{d} | 29.694 ± | 0.205^{d} |
| CW21-4 | 9.678 ± | 0.067^{b} | 23.059 ± | 0.054^{c} | 32.737 ± | 0.118^{c} |
| CW21-5 | 14.979 ± | 0.219^{h} | 38.099 ± | 0.027^{a} | 53.078 ± | 0.242^{g} |
| CW21-6 | 0.530 ± | 0.001^{k} | 5.650 ± | 0.006^{l} | 6.181 ± | 0.005^{m} |
| CW21-7 | 1.924 ± | 0.010^{g} | 14.383 ± | 0.006^{g} | 16.307 ± | 0.015^{h} |
| CW21-8 | 0.966 ± | 0.004^{l} | 8.595 ± | 0.002^{l} | 9.560 ± | 0.003^{k} |
| CW21-9 | 1.143 ± | 0.032^{h} | 14.806 ± | 0.005^{f} | 15.949 ± | 0.037ⁱ |
| CW21-10 | 2.750 ± | 0.013^{f} | 15.106 ± | 0.003^{e} | 17.855 ± | 0.015^{f} |
| CW21-11 | 0.908 ± | 0.004^{f} | 3.487 ± | 0.003^{m} | 4.395 ± | 0.003^{o} |
| CW21-12 | 4.187 ± | 0.021^{e} | 14.860 ± | 0.006^{f} | 19.048 ± | 0.015^{e} |
| CW21-13 | 4.193 ± | 0.027^{e} | 12.337 ± | 0.024^{h} | 16.529 ± | 0.025^{g} |
| CW21-14 | 0.654 ± | 0.001^{f} | 5.675 ± | 0.002^{f} | 6.330 ± | 0.002^{m} |
| CW21-15 | 1.881 ± | 0.020^{g} | 3.290 ± | 0.002ⁿ | 5.171 ± | 0.021ⁿ |
| CW21-16 | 1.254 ± | 0.006^{b} | 7.884 ± | 0.004^{k} | 9.138 ± | 0.002^{l} |

Different alphabets indicate differences in DMRT (Duncan's Multiple Range Test) at the 5% significance level (p < 0.05)

On Day 50 after the flowering, a comprehensive investigation of morphology and compositional correlations was conducted for all hemp F₁ generations. Heatmap analysis of plant height, number of branches, and the ratio of cannabinoids in leaves (total CBD/total Δ9-THC) revealed the formation of one cluster group for stem diameter, leaf length, and leaf width. Furthermore, the cannabinoid ratio of inflorescence (total CBD/total Δ9-THC), total CBD concentration in inflorescence, total CBD concentration in leaves, and total Δ9-THC concentration in leaves formed another single cluster group, while the red color ratio of leaf stems, red color ratio of stems, total Δ9-THC concentration in inflorescence, inflorescence number, and plant constituted a still another cluster. According to the heatmap analysis, the total CBD content and plant diameter exhibited a strong inverse relationship in CW21-5. Thus, CW21-5 was evaluated to occupy less space but possess higher cannabinoid content, thereby having a characteristic of potentially increasing the total CBD production amount per unit area. Meanwhile, since many morphological features and cannabinoid contents were classified into different clusters to represent minimal or absolutely no correlation, principal component analysis (PCA) was conducted for more accurate results. PCA was performed using MetaboAnalyst 5, and hierarchical cluster analysis utilized the Euclidean distance algorithm.

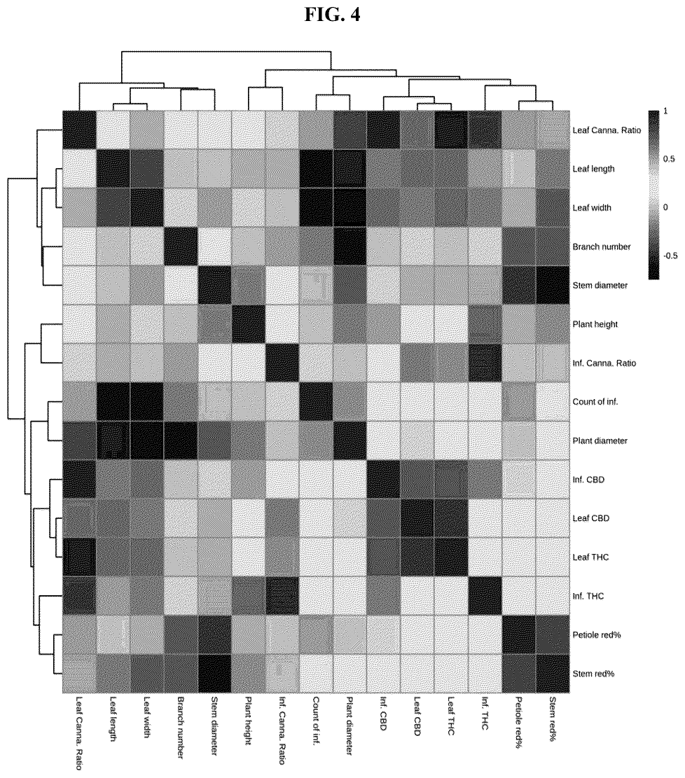

According to the PCA heatmap results, it was found that the number of flowers and the diameter of plants are naturally proportional (FIG. 4). Many morphological data, such as stem diameter and leaf length, showed a tendency to be inversely proportional to the plant diameter. Most morphological data and cannabinoid contents did not exhibit significant correlations, except for the THC content in inflorescence, and all cannabinoids were found to be proportional to each other. The percentage of reddishness in stems and leaf petioles showed an inverse relationship with stem diameter. In summary, based on these results, the correlation between cannabinoid content and morphology was deemed to be very low.

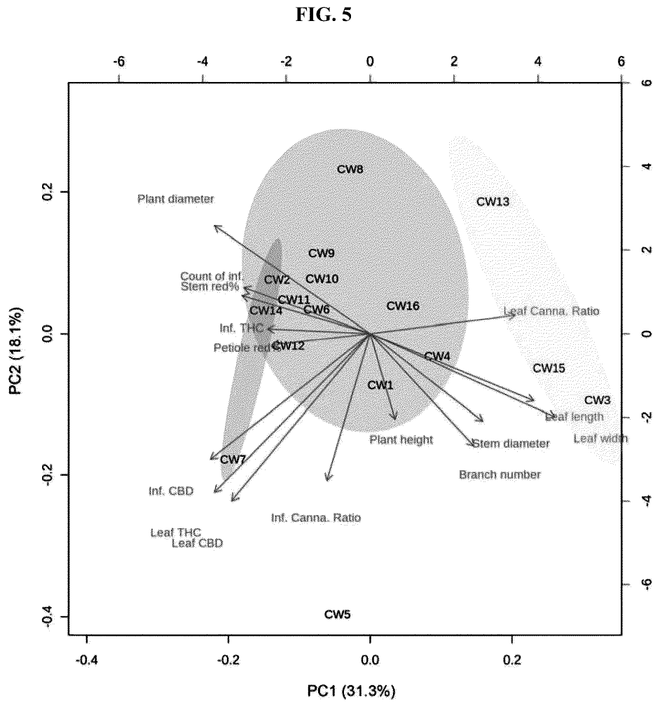

Furthermore, based on the self-organizing map (SOM) clustering of the PCA graph, it was found that CW21-2, CW21-7, and CW21-14 formed the first cluster. CW21-1, CW21-2, CW21-4, CW21-6, CW21-8, CW21-9, CW21-10, CW21-11, CW21-12, CW21-14, and CW21-16 formed the second cluster, while CW21-3, CW21-13, and CW21-15 formed the third cluster, and CW21-5 was found to not belong to any cluster (FIG. 5). As a result of analyzing CW21-5 based on the PC2 axis, a negative correlation of the plant diameter with CW21-5 and a positive correlation with cannabinoid amount, except for the total Δ9-THC concentration in the inflorescence, were shown. The cannabinoid ratio (total CBD/total Δ9-THC) in the inflorescence also indicated a high ratio, again showing the superiority of CW21-5 that is characterized by low total Δ9-THC content and high total CBD content in the inflorescence.

The cannabinoid content analysis results for the Cherry wine as the seed parent of CW21-5 after grown under the same conditions as above are shown in Table 8. It was found that the CW21-5 line exhibits, in both leaves and inflorescence, higher CBD and CBDA contents compared to the seed parent.

TABLE 8
Cannabinoid content in Cherry wine

| | | Cannabinoid content (%) | | | |
|---|---|---|---|---|---|
| Sample name | Type | CBDA | CBD | Δ9-THCA | Δ9-THC |
| Cherry wine | Inflorescence | 2.45810 | 0.06228 | 0.02759 | 0.04297 |
| | Leaf | 0.47239 | 0.00500 | 0.14066 | 0.00636 |

### Example 3. Physiological Activity Analysis of CW21-5 as Medical Hemp

The inventors of the present invention analyzed the physiological activity of the CW21-5 line selected in the above. Briefly, the CW21-5 line and the Chungsam cultivar (i.e., hemp variety for obtaining fiber and seeds) were cultivated in a greenhouse with temperature control and shading. Flowering was induced through short-light treatment, and after 50 days, 10 leaves were collected from the top, middle, and bottom of the plants. The leaves were freeze-dried, crushed, and homogenized to yield a homogeneous mixture. Inflorescence samples were also collected in the same manner. 0.1 g of the crushed sample was weighed and mixed with 80% methanol (DAEJUNG Chemicals and Metals Co., Ltd., Korea) in an amount of 1000 times the sample. The resulting mixture was then sonicated for 40 minutes at room temperature. Subsequently, it was concentrated at 30° C. using a rotary evaporator (Eyela N-1000, Tokyo Rikakikai Co., Ltd., Japan) and used for the following tests.

### 3-1. Anticancer Activity

Human lung cancer cells (A549) were prepared in a 96-well plate at a concentration of 5×10⁴ cells/ml. Extract of the CW21-5 line or the Chungsam cultivar was then treated at concentrations of 1,000, 500, 250, 125, 63, 31, 16, or 8 μg/ml and allowed to react for 12 or 24 hours. Subsequently, the viability of lung cancer cells was assessed using MTT (3-(4,5-dimethylthiazol-2-yl)-2,5-diphenyltetrazolium bromide) analysis.

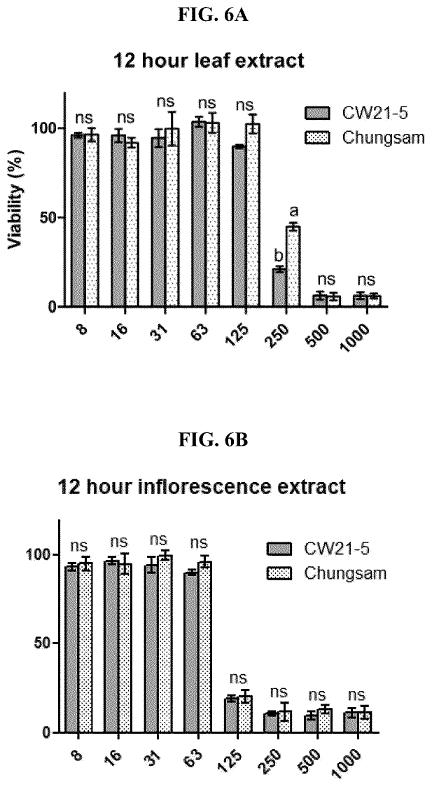

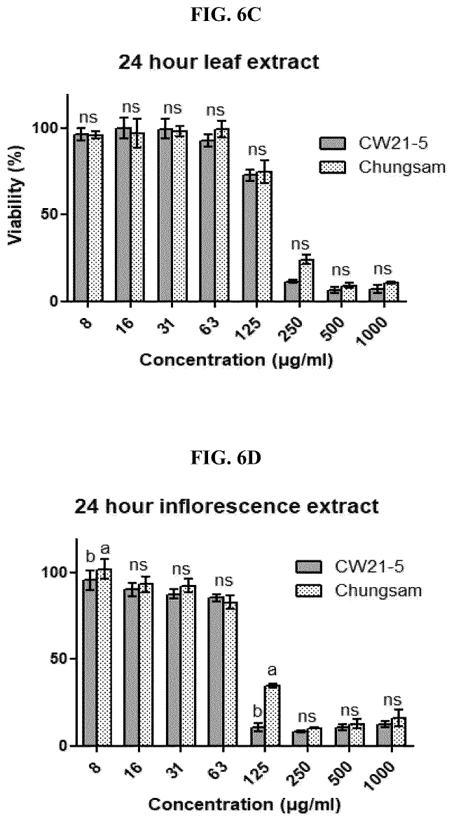

The results indicated that inflorescence extracts from both the CW21-5 line and the Chungsam cultivar exhibited better cytotoxic effects on lung cancer cells than the leaf extracts. Additionally, extracts from the CW21-5 line showed superior cytotoxic effects on cancer cells compared to extracts from the Chungsam cultivar (FIGS. 6A to 6D).

### 3-2. Antimicrobial Activity

The indicator lines used to determine the antimicrobial activity of the hemp extracts are as follows: Escherichia coli (KCTC1924), Staphylococcus aureus (KCTC1916), Klebsiella pneumonia (KCTC2001), Salmonella typhimurium (KCTC1925), and Streptococcus mutans (KCTC5365). The antimicrobial activity of CW21-5 line or Chungsam cultivar extracts was analyzed in terms of the minimum Inhibitory Concentration (MIC).

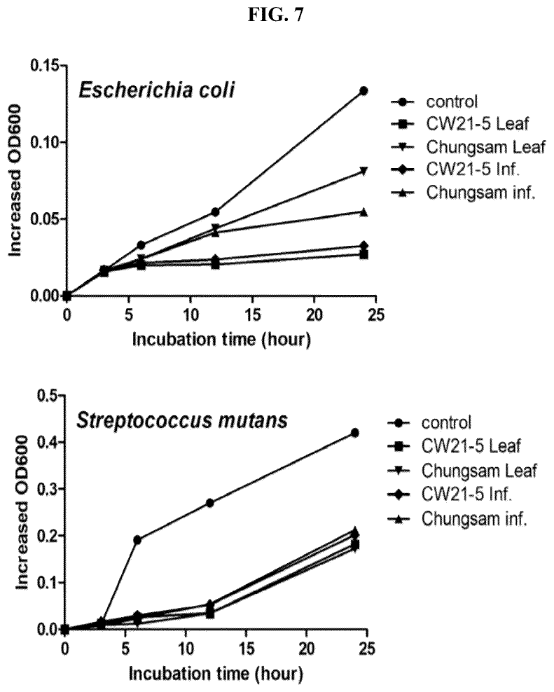

As a result of the analysis of minimum inhibitory concentration for each extract, it was found that the leaf and inflorescence extracts of both CW21-5 and Chungsam cultivar did not show any antimicrobial activity against Staphylococcus aureus, Klebsiella pneumonia, and Salmonella typhimurium (Table 9). However, the CW21-5 extract exhibited superior antimicrobial activity against Escherichia coli compared to the Chungsam extract (FIG. 7).

TABLE 9
Result of minimum inhibitory concentration analysis

| | | Minimum inhibitory concentration (μg/ml) | | | | |
|---|---|---|---|---|---|---|
| | | Gram positive | | Gram negative | | |
| Type of samples | | S. aureus | S. mutans | E. coli | K. pneumonia. | S. typhimurium |
| CW21-5 | Leaf | ND²⁾ | ≤12.50 | ≤1.56 | ND | ND |
| | Inf.¹⁾ | ND | ≤6.25 | ≤1.56 | ND | ND |
| Chungsam | Leaf | ND | ≤6.25 | ≤3.13 | ND | ND |
| | Inf. | ND | ≤12.50 | ≤3.13 | ND | ND |

The concentration was selected to perfectly inhibit the growth of microorganisms.
¹⁾Inf.; inflorescence, ²⁾ND; not detected.

The inventors of the present invention named the CW21-5 line, which demonstrates high CBD productivity in the aforementioned smart farm cultivation and exhibits higher antimicrobial and anti-lung cancer activity compared to existing Korean cultivation varieties, as Cherry king (Cannabis sativa L. cv. Cherry king).

What is claimed is:

1. A hemp cultivar Cherry king (Cannabis sativa L. cv. Cherry king) plant obtained through open pollination using Cherry wine genetic resource as a seed parent, the hemp cultivar Cherry king plant having a higher content of cannabinoids than the seed parent, the hemp cultivar Cherry king plant deposited under Accession Number KACC 88007BP.

2. A progeny plant derived from the hemp cultivar Cherry king plant of claim 1.

3. A feminized seed obtained by induction of male flowers on female plant of the hemp cultivar Cherry king plant of claim 1.

4. Hempseed oil or a seed protein derived from the feminized seed of claim 3.

5. A F₁ hemp plant produced by crossing the hemp cultivar Cherry king plant of claim 1 with other hemp cultivar.

6. A seed of the F₁ hemp plant according to claim 5.

7. Hempseed oil or a seed protein derived from the seed of the F₁ hemp plant of claim 6.

8. A transgenic hemp plant obtained by transformation of the hemp cultivar Cherry king plant of claim 1.

9. A mutant plant obtained by treating the hemp cultivar Cherry king plant of claim 1 with mutagen.

10. A functional health food composition comprising an extract of the hemp cultivar Cherry king plant according to claim 1 as effective component.

11. A pharmaceutical composition comprising an extract of the hemp cultivar Cherry king plant according to claim 1 as effective component.

12. An antimicrobial composition comprising an extract of the hemp cultivar Cherry king plant according to claim 1 as effective component.

13. A cosmetic composition comprising an extract of the hemp cultivar Cherry king plant according to claim 1 as effective component.
